(19) United States
(12) Patent Application Publication
(10) Pub. No.: US 2014/0363507 A1
(43) Pub. Date: Dec. 11, 2014

(54) DENTRIC POLYGLYCEROL SULFATES AND SULFONATES AND THEIR USE FOR INFLAMMATORY DISEASES

(71) Applicant: FREIE UNIVERSITAT BERLIN, BERLIN (DE)

(72) Inventors: Rainer HAAG, Berlin (DE); Jens DERNEDDE, Berlin (DE); Rudolf TAUBER, Berlin (DE); Gesche BERNHARD, Baltschieder (CH); Sven ENDERS, Berlin (DE); Heidemarie WEINHART, Berlin (DE); Arne VON BONIN, Glienicke/Nordbahn (DE); Ulrich ZÜGEL, Berlin (DE); Holger TÜRK, Mannheim (DE)

(73) Assignee: FREIE UNIVERSITAT BERLIN, BERLIN (DE)

(21) Appl. No.: 14/469,095

(22) Filed: Aug. 26, 2014

Related U.S. Application Data
(63) Continuation of application No. 12/376,174, filed on Nov. 16, 2009.

(30) Foreign Application Priority Data
Aug. 3, 2006 (DE) 10 2006 036 326.4

(51) Int. Cl. C07C 305/10; C08G 65/48; C07K 14/00

(52) U.S. Cl. CPC C07C 305/10; C07K 14/00; C08G 65/48
USPC 424/484; 558/26; 562/103; 530/395; 530/402; 424/78.17; 514/711; 435/7.92; 525/535

(57) ABSTRACT

The present invention relates to the novel compound classes of dendritic polyglycerol sulfates and sulfonates as well as to their production and use for the treatment of diseases, particularly inflammatory diseases, and to their use as selectin inhibitors and selectin indicators. The dendritic polyglycerol sulfates and sulfonates are also suitable for imaging diagnostics, particularly with respect to inflammatory diseases.

The present invention relates to the novel compound classes of dendritic polyglycerol sulfates and sulfonates as well as to their production and use for the treatment of diseases, particularly inflammatory diseases, and to their use as selectin inhibitors and selectin indicators. The dendritic polyglycerol sulfates and sulfonates are also suitable for imaging diagnostics, particularly with respect to inflammatory diseases.

### BACKGROUND OF THE INVENTION

Inflammation is a fundamental response to the damage of tissue and the invasion of pathogens, wherein leukocytes play a key role due to their antimicrobial, secretory and phagocytosis activities. Recruiting of leukocytes to the vascular endothelium and subsequent migration into the surrounding tissue are observed in all forms of the inflammatory reaction.

The migration of leukocytes into tissue is initiated by the adherence of leukocytes onto the vascular wall. This allows the leukocytes to accumulate in the source of infection and to effect defense reactions. A variety of vascular cell adhesion molecules on leukocytes and on endothelium cells mediate and control the adhesion of blood cells onto the vascular wall. This process takes place in a cascade of series connected molecular interactions. At first, selectins, a family of lectin-like adhesion molecules, mediate the “docking” and rolling of the leukocytes on the surface of the endothelium. This leads to a slowdown of the leukocytes and allows the contact with signalling molecules on the surface of the endothelium, like e.g. chemokins. These signalling molecules stimulate the activation of integrins on the surface of leukocytes which than in turn mediate the efficient binding of these cells onto the surface of the endothelium. Members of the superfamily of immunoglobulins (Ig) act as ligands of the integrins. The now stably adherent leukocytes can move in a directed manner and can actively move through the endothelium cell layer.

As already stated, the initial contact and the rolling of the leukocytes on the endothelium is mediated by transient receptor-ligand interactions between the (three) selectins and their ligands [1]. Close contact of the leukocytes with the endothelium is subsequently guaranteed via the interaction of activated integrins with adhesion molecules of the immunoglobuline (Ig) super family [2]. In addition to the desired defense action and the repair of tissue damages the uncontrolled migration of leukocytes from the bloodstream can be of pathological relevance and lead to the damage of tissue [3]. The general attendance of endothelial cell adhesion molecules in acute and chronic inflammatory processes renders them suitable target structures for diagnostics and therapy [for a review see 4].

Selectins are carbohydrate binding adhesion molecules, which contribute to the increased adhesion of leukocytes onto the vascular endothelium of the inflamed tissue during the process of immune defense. According to their cells of origin, they are divided into L-(leukocytes), E-(endothelium) and P-selectin (platelets and endothelium). Due to their protein structure and their special molecular binding characteristics selectins initiate leukocyte adhesion; after temporarily binding of the corresponding ligands the leukocytes experience a “rolling slowdown” from the fluent bloodstream alongside the vascular wall. Afterwards other adhesion molecules mediate the close binding of the leukocytes onto the endothelium as well as their extravasation for accomplishing their defense function. Shortly after the discovery of selectins and after the elucidation of their structure at the beginning of the nineties the selectins became attractive target structures in the field of pharmaceutical research. In addition to their physiological function in immune response, a dysregulation of the selectin expression during pathological processes, such as rheumatoid arthritis, asthma, diabetes mellitus and ischemia/reperfusion, as well as an attendance during the tissue invasion of metastasizing cancer cells was observed. This motivated an intensive search for selectin inhibiting compounds.

E- and P-selectin, and L-selectin ligands are expressed on microvascular endothelium in an inflammation-dependent manner, L-selectin is presented on leukocytes [1, 2]. Only several highly affine ligands are known for the reported selectins. In principle, these are mucin-like structures, i.e. long elongated glycoproteins, which have many carbohydrate side chains glycosidically attached on their serine or threonine rich protein scaffold as the actual binding epitopes. Via fast formation and dissociation of receptor bindings on the highly flexible ligands cell rolling is mediated in the shearing stream of the vessels. The carbohydrate epitopes essential for binding are N-acetyl lactosamin based oligosaccharides with a specifically attached fucose and a terminal sialic acid (N-acetyl neuraminic acid). The tetrasaccharide sialyl LewisX (sLeX) is an outstandingly relevant binding epitope, sLeX is used as a standard ligand for structure-function relations in order to characterize binding characteristics as well as for searching selectin inhibitors.

The findings of sLex as an important binding partner of selectins and findings of polyvalence as a key for targeted blockage of leukocyte adhesion are known for quiet some time and are the basis for the development of diverse selectin inhibitors [for a review see 5]. As of yet, the target selectin has not led to the development of market-ready therapeutics, even though highly affine inhibitors are available [5].

At present therapeutic intervention in the case of rheumatoid arthritis and psoriasis is achieved by employing blockers of the inflammatory cytokin TNFα (infliximab, etanercept, [6,7]). In addition, with efalizumab [7] an anti-integrin antibody is on the market, which is approved for the systemic therapy of psoriasis. Further compounds are tested in clinical trials, such as the Pan-selectin antagonist bimosiamose (1,6-bis[3-(3-carboxymethylphenyl)-4-(2-alpha-D-mannopyranosyloxy)phenyl]hexane) which belongs to the class of small molecule drugs and which is a selectin inhibiting compound with a glycoside structure having a substantially higher affinity to selectins than sLeX (trials performed by Revotar AG, Hennigsdorf). Bimosiamose is supposed to be employed for asthma, psoriasis, atopical dermatitis and reperfusion damages [8].

Linear neoglycopolymers carrying sulfated sLex structures have been described and can reach IC₅₀ values in the low nanomolecular range [5, 9], as well as dendritic polyethylene oxide (PEO) glycopolymers, which are sulfated [10].

Therefore, it is an object of the present invention to provide compounds and compound classes, which are easy to be synthesized and which are suitable for the treatment of diseases, particularly inflammatory diseases.

The object is solved by the present invention by providing dendritic polyglycerol sulfonates.

A dendritic polyglycerol sulfonate according to the present invention is characterized by
a) a polymeric polyglycerol core, composed of repeated units of glycerin with the formula (RO—CH₂)₂CH—OR
   on a multifunctional starter molecule, which is a polyhydroxy compound having 1 to 1,000 OH groups, preferably 1 to 4 OH groups,
      wherein R═H or further glycerin units,
   the core having a branching degree of 0 to 100%, preferably 60%, and
   an average molecular weight of 100 to 1,000,000 g/mol, preferably 1,000 to 20,000 g/mol,
b) the substitution of one or more OH groups of the glycerin units with —SO₃H or —SO₃Na groups
   or the attachment of an oligomeric spacer at one or more OH groups of the glycerin units,
   the oligomeric spacer having the generic formula
   —(CH₂)ₙ— or —[(CH₂)ₘ—O)]ₙ—, wherein m is 1 to 100 and n is 1 to 50,000, and bound thereto —SO₃H or —SO₃Na groups,
   so that a degree of sulfonation of 1 to 100%, preferably 1 to 30%, is obtained,
and
c) a molecular weight of 110 to 1,500,000 g/mol, preferably 1,100 to 30,000 g/mol.

The polymeric polyglycerol core is produced by using a (multi)functional starter molecule or initiator, respectively, during the ring-opening polymerization of glycidol. The starter molecule or initiator, respectively, is a polyhydroxy compound, having 1 to 1,000, preferably 1 to 100 and more preferably 1 to 4 OH groups. The starter molecule has the generic formula R—(OH)ₓ, wherein R can be any molecule, which is stable under the conditions of the anionic polymerization, and x is 1 to 1,000; preferably 1 to 100 and more preferably 1 to 4. Preferably the used initiators are tris- or tetrafunctional initiators, such as 1,1,1-trishydroxymethylpropane (TMP) as preferred trisfunctional initiator or pentaerythrol (PE) as preferred tetrafunctional initiator. The starter molecule or the initiator, respectively, can carry further heterofunctionalities, such as particularly SH groups, NH₂ groups. In a particular embodiment the starter molecule contains OH groups and/or further heterofunctionalities (like SH, NH₂). Further suitable initiators are known to the person of skill in the art.

Depending on the choice of the initiator and the polymerization conditions the polymeric polyglycerol core reaches a branching degree and an arbitrarily adjustable molecular weight with narrow polydispersities. According to the present invention polymeric polyglycerol cores with a branching of 0 to 100% are used. Preferably, highly branched structures are used, preferably with a branching degree of 30 to 80%, particularly preferably with a branching degree of 60%.

The average molecular weight of the polymeric polyglycerol core according to the present invention is preferably 100 to 1,000,000 g/mol, more preferably 500 to 100,000 g/mol, wherein 1,000 to 20,000 g/mol are particularly preferred.

The polymeric polyglycerol cores according to the present invention are subjected to a sulfonation. Preferably sodium salt of vinylsulfonic acid in presence of catalytic amounts of a base, such as potassium hydroxide, is used as sulfonation reagent. The degree of sulfonation reached is preferably 1 to 100%, particular preferably 10 to 30%, more particular preferably 30 to 100%.

“Degree of sulfonation” according to this invention means the percentage of functionalized OH groups of the glycerine units of the polymeric polyglycerol core. The functionalization results either from the substitution of one or more OH groups of the glycerin units with —SO₃H or —SO₃Na groups or from the attachment of an oligomeric spacer at one or more OH groups of the glycerin units.

The oligomeric spacer has the generic formula:

—(CH₂)ₙ—

or

—[(CH₂)ₘ—O)]ₙ—,
wherein m is 1 to 100, preferably 1 to 50, more preferably 1 to 10 and even more preferably 2, and
n is 1 to 50,000, preferably 1 to 5,000, more preferably 1 to 100 and has bound thereto —SO₃H or —SO₃Na groups.

An oligomeric spacer is e.g. a oligoethylene glycol (OEG) chain, a polyethylene glycol (PEG) chain, aliphatic carbohydrate chains or also other linear polyethers.

Depending on the choice of the polymeric polyglycerol cores according to the present invention and the sulfonation conditions, i.e. the degree of sulfonation, the molecular weight of a dendritic polyglycerol sulfonate according to the present invention is preferably 110 to 1,500,000 g/mol, more preferably 600 to 150,000 g/mol and particular preferably 1,100 to 30,000 g/mol.

Particularly preferred embodiments of a dendritic polyglycerol sulfonate according to the present invention have

a) a polymeric polyglycerol core with an average molecular weight (Mₙ) of 2,500 to 20,000 g/mol and a branching degree of 60%, which corresponds to a dendritic branching degree, and
b) a degree of sulfonation of 10 to 30%, which is obtained by sulfonation with sodium salt of vinylsulfonic acid.

A particularly preferred embodiment of a dendritic polyglycerol sulfonate according to the present invention has a polymeric polyglycerol core with an average molecular weight of 5,000 g/mol, a degree of sulfonation of 4% and a molecular weight of 5,200 g/mol, such as compound 3b of Example 2 (see Table 2).

A further particularly preferred embodiment of a dendritic polyglycerol sulfonate according to the present invention has a polymeric polyglycerol core with an average molecular weight of 20,000 g/mol, a degree of sulfonation of 8% and a molecular weight of 21,800 g/mol, such as compound 3d of Example 2 (see Table 2).

The object is furthermore solved by the present invention by providing dendritic polyglycerol sulfates.

A dendritic polyglycerol sulfate according to the present invention is characterized by:
a) a polymeric polyglycerol core, composed of repeated units of glycerin with the formula (RO—CH₂)₂CH—OR
   on a multifunctional starter molecule, which is a polyhydroxy compound having 1 to 1,000 OH groups, preferably 1 to 4 OH groups,
      wherein R═H or further glycerin units,
   the core having a branching degree of 0 to 100%, preferably 60%, and
   an average molecular weight of 100 to 1,000,000 g/mol, preferably 1,000 to 20,000 g/mol, more preferably 2,000 to 7,500
b) the substitution of one or more OH groups of the glycerin units with —OSO₃H or —OSO₃Na groups
   or the attachment of an oligomeric spacer at one or more OH groups of the glycerin units,
   the oligomeric spacer having the generic formula
   —(CH₂)ₙ— or —[(CH₂)ₘ—O)]ₙ—, wherein m is 1 to 100 and n is 1 to 50,000, and bound thereto —OSO₃H or —OSO₃Na groups,
   so that a degree of sulfation of 1 to 100% is obtained,
and
c) a molecular weight of 200 to 5,000,000 g/mol, preferably 2,000 to 50,000 g/mol, more preferably 5,000 to 13,500.

The polymeric polyglycerol core is produced by using a (multi)functional starter molecule or initiator, respectively, during the ring-opening polymerization of glycidol. The starter molecule or initiator, respectively, is a polyhydroxy compound, having 1 to 1,000, preferably 1 to 100 and more preferably 1 to 4 OH groups. The starter molecule has the generic formula R—(OH)ₓ, wherein R can be any molecule, which is stable under the conditions of the anionic polymerization, and x is 1 to 1,000; preferably 1 to 100 and more preferably 1 to 4. Preferably the used initiators are tris- or tetrafunctional initiators, such as 1,1,1-trishydroxymethylpropane (TMP) as preferred trisfunctional initiator or pentaerythrol (PE) as preferred tetrafunctional initiator. The starter molecule or the initiator, respectively, can carry further heterofunctionalities, such as particularly SH groups, NH₂ groups. In a particular embodiment the starter molecule contains OH groups and/or further heterofunctionalities (like SH, NHa). Further suitable initiators are known to the person of skill in the art.

Depending on the choice of the initiator and the polymerization conditions the polymeric polyglycerol core reaches a branching degree and an arbitrarily adjustable molecular weight with narrow polydispersities. According to the present invention polymeric polyglycerol cores with a branching of 0 to 100% are used. Preferably, highly branched structures are used, preferably with a branching degree of 30 to 80%, particularly preferably with a branching degree of 60%.

The average molecular weight of the polymeric polyglycerol core according to the present invention is preferably 100 to 1,000,000 g/mol, more preferably 500 to 100,000 g/mol, wherein 1,000 to 20,000 g/mol as well as 2,000 to 7,500 are particularly preferred.

The polymeric polyglycerol cores according to the present invention are subjected to a sulfation. Preferably a complex of SO₃ and pyridine is used as sulfation reagent, and in a concentration that is equimolar to the OH groups of the polymeric polyglycerol core. The resulting functionalization, i.e. sulfation, of 0 to 100% can be adjusted via the ratio of SO₃ to the OH groups of the polyglycerol. The degree of sulfation reached is preferably 1 to 100%, particular preferably 70 to 95%, more particular preferably 75 to 92%.

“Degree of sulfation” according to this invention means the percentage of functionalized OH groups of the glycerine units of the polymeric polyglycerol core. The functionalization results either from the substitution of one or more OH groups of the glycerin units with —OSO₃H or —OSO₃Na groups or from the attachment of an oligomeric spacer at one or more OH groups of the glycerin units.

The oligomeric spacer has the generic formula:

—(CH₂)ₙ—

or

—[(CH₂)ₘ—O)]ₙ—,
wherein m is 1 to 100, preferably 1 to 50, more preferably 1 to 10 and even more preferably 2, and
n is 1 to 50,000, preferably 1 to 5,000, more preferably 1 to 100 and has bound thereto —OSO₃H or —OSO₃Na groups.

An oligomeric spacer is e.g. a oligoethylene glycol (OEG) chain, a polyethylene glycol (PEG) chain, aliphatic carbohydrate chains or also other linear polyethers.

Depending on the choice of the polymeric polyglycerol cores according to the present invention and the sulfation conditions, i.e. the degree of sulfation, the molecular weight of a dendritic polyglycerol sulfate according to the present invention is preferably 200 to 5,000,000 g/mol, more preferably 2,000 to 50,000 g/mol and particularly preferable 5,000 to 13,500, particular preferably 5,500 g/mol or 11,000 g/mol or 21,500 g/mol or 41,000 g/mol or 6,800 g/mol or 8,600 g/mol or 12,300 g/mol.

A particularly preferred embodiment of a dendritic polyglycerol sulfate according to the present invention has a polymeric polyglycerol core with an average molecular weight of 2,500 g/mol, a degree of sulfation of 85% and a molecular weight of 5,500 g/mol, such as compound 2a of Example 1 (see Table 1) or compound dPGS250085 of Example 4 (see Table 3), respectively.

A further particularly preferred embodiment of a dendritic polyglycerol sulfate according to the present invention has a polymeric polyglycerol core with an average molecular weight of 5,000 g/mol, a degree of sulfation of 79% and a molecular weight of 10,500 g/mol, such as compound 2b of Example 1 (see Table 1).

A further particularly preferred embodiment of a dendritic polyglycerol sulfate according to the present invention has a polymeric polyglycerol core with an average molecular weight of 2,500 g/mol, a degree of sulfation of 92% and a molecular weight of 6,800 g/mol, such as compound dPGS2500/92 of Example 4 (see Table 3).

A further particularly preferred embodiment of a dendritic polyglycerol sulfate according to the present invention has a polymeric polyglycerol core with an average molecular weight of 4,000 g/mol, a degree of sulfation of 84% and a molecular weight of 8,600 g/mol, such as compound dPGS4000/84 of Example 4 (see Table 3).

A further particularly preferred embodiment of a dendritic polyglycerol sulfate according to the present invention has a polymeric polyglycerol core with an average molecular weight of 6,000 g/mol, a degree of sulfation of 76% and a molecular weight of 12,300 g/mol, such as compound dPGS6000/76 of Example 4 (see Table 3).

The object is furthermore solved by the use of a dendritic polyglycerol sulfonate according to the present invention and/or a dendritic polyglycerol sulfate according to the present invention as medicament for the treatment of diseases.

The compounds according to the present invention can be provided, for example, when used as medicaments, in form of pharmaceutical compositions, which comprise one or more of the compounds of the present invention as well as pharmaceutical acceptable carriers. Preferably, these pharmaceutical compositions have a unit dosage form, such as tablets, pills, capsules, powder, granulate, sterile parenteral solutions or suspensions. Further dosage forms are known to the person of skill in the art.

A medicament or a pharmaceutical composition comprises a therapeutically effective amount of the drug or of several drugs, i.e. a therapeutically effective amount of one or more compounds of the present invention. A skilled person will be able to determine the therapeutically effective amount on the basis of the disease to be treated and in consideration of the state of the patient. A medicament or a pharmaceutical composition can suitably contain between about 5 and 1000 mg, preferably about 10 to 500 mg of a compound according to the present invention.

The pharmaceutical acceptable carrier and/or excipient can have a wide variety of forms depending on the desired route of application (e.g. oral, parenteral). Suitable carrier and excipients are known in the art and can be selected by a person of skill in the art. Carrier include inert pharmaceutical excipients, like binding agents, suspension agents, lubricants, flavoring agents, sweetener, preservative agents, coloring agents and coating agents.

The diseases, which can be treated by using a dendritic polyglycerol sulfonate according to the present invention and/or a dendritic polyglycerol sulfate according to the present invention, are preferably inflammatory diseases.

For a therapeutic intervention all inflammatory processes are considered, wherein the migration of the leukocytes from the bloodstream is pathologically relevant and results in tissue damage. Besides the chronic inflammatory diseases, such as e.g. rheumatoid arthritis, asthma and psoriasis, the use of the dendritic polyglycerol sulfonates according to the present invention and/or the dendritic polyglycerol sulfates according to the present invention is also possible in case of ischemia reperfusion damages or graft repulsion.

The compounds according to the present invention are, thus, preferably used for the treatment of chronic inflammatory diseases, particularly rheumatoid arthritis, asthma and psoriasis, as well as for the treatment of ischemia reperfusion damages or graft repulsion.

More preferably, the inflammatory diseases are diseases, wherein the selectin-mediated leukocyte adhesion is dysregulated.

The antiinflammatory effect of the compounds of the invention can, for example, be attributed to the reduction of leucocyte emigration mediated by them. As a result activation of further immune cells by secreted cytokines at the site of inflammation is greatly reduced. (for further details see Examples).

In chronic inflammatory processes, fibrosis develop subsequent to tissue damage. Thereby, two cytokines play an important role: IFNγ and TNFα. IFNγ is secreted from a particular population of leukocytes (T and NK cells) and leads to an activation of macrophages, which in turn:

1) produce hydrolytic enzymes and reactive oxygen and nitrogen species, which leads to a destruction of the adjacent tissue, and
2) release TNFα, which leads to an increased expression of cell adhesion molecules on adjacent endothelia and an activation of leukocytes.

Therefore, an increased recruiting of leukocytes is observed in inflammation areas.

The object is furthermore solved by the use of a dendritic polyglycerol sulfonate according to the present invention and/or a dendritic polyglycerol sulfate according to the present invention as selectin inhibitor.

Preferred is thereby the use as inhibitor of L-selectin and/or P-selectin.

The dendritic polyglycerol sulfonates according to the present invention and/or the dendritic polyglycerol sulfates according to the present invention bind L- and P-selectin with high affinity (IC₅₀ of 10 nM or 40 nM, respectively, see Example 3) and, thus, block the interaction with their ligands. The leukocyte-endothelium contact is reduced and, thus, the increased migration of the leukocytes into the inflammation sites is suppressed.

The dendritic polyglycerol sulfonates according to the present invention and/or polyglycerol sulfates are therefore suitable for the inhibition of the selectin-mediated leukocyte adhesion.

The object is furthermore solved by the use of a dendritic polyglycerol sulfonate according to the present invention and/or a dendritic polyglycerol sulfate according to the present invention as selectin indicator.

A “selectin indicator” according to the present invention binds specifically to selectins or one of the selectins, like L- and/or P-selectin, and can, thus, be used for targeting, localizing and/or visualizing the selectins, particularly in vitro, in cells, in tissue, in organs, in tissue sections but particularly also in vivo. By applying the teaching of this patent application, the skilled person will be able to use the compounds according to the present invention as selectin indicators.

For this purpose, the compounds of the present invention will preferably be loaded with signalling molecules or signalling molecules will be bound to the compounds of the present invention.

Preferred signalling molecules are molecules labelled with radioactive isotopes, such as ¹²⁴I, ¹²⁵I, or ¹⁸F, molecules labelled with dyes, particularly fluorophores, such as aminomethyl coumarin, fluorescein, cyanine, rhodamine and their derivatives, or other chromophores. A signalling molecule can further be a fluorescence donor or reporter and a fluorescence acceptor or quencher, which can particularly be used as a pair of each a fluorescence donor/reporter and a fluorescence acceptor/quencher (i.e. as a FRET pair).

So far the localization and characterization of inflammation sources is not satisfactory solved by the available methods of imaging clinical diagnostics. For a specific targeting of inflammation areas with the dendritic polyglycerol sulfonates according to the present invention and/or the dendritic polyglycerol sulfates according to the present invention these compounds are loaded with signal donors (radioisotopes, NIR dyes, magnetit) and used for a visualization. Requirements therefore are a specific binding (to L- and P-selectin) and accumulation of the signal at the inflammation area.

Accordingly, the compounds of the present invention are preferably used for the diagnosis of inflammatory diseases. Thereby, a targeting of the selectins occurs in the area of the inflammation.

The dendritic polyglycerol sulfonates according to the present invention and/or the dendritic polyglycerol sulfates according to the present invention act furthermore as heparin analog and are, thus, like heparin, able to specifically bind some of the chemokines. These chemokines are proinflammatory cytokines, particularly TNFα, IL-1, IL-6, as well as IL-8 and MIP-1β.

An inhibitory binding of the chemokines, like INFγ or TNFα, by a dendritic polyglycerol sulfonate according to the present invention and/or a dendritic polyglycerol sulfate according to the present invention prevents an interaction with the receptors of the chemokines, which results in reduced tissue damage and leukocyte extravasation.

Due to their specific interactions with proteins, like selectins, chemokines, coagulation factors, particularly L- and P-selectin, the compounds of the present invention are preferably used in further in vitro applications:
The dendritic polyglycerol sulfonates according to the present invention and/or the dendritic polyglycerol sulfates according to the present invention are (analogous to commercially available heparin sepharose) immobilized on a matrix.

Preferred matrices or surfaces for immobilizing, respectively, are inorganic as well as polymeric natural and synthetic materials depending on the use, for example the separation technique used. Examples are silicon-based surfaces (e.g. glass, silica) and various functionalized and non-functionalized polymers (e.g. dextran, agarose, sepharose as well as synthetic hydrophilic polymers).

The matrices or surfaces for immobilizing, respectively, are furthermore selected from the group consisting of inorganic oxide surfaces, magnetizable or non-magnetizable surfaces, silicon containing surfaces, glass surfaces, silica membranes, siliceous earths, clays and further surfaces that are known to the skilled artisan. The matrices or surfaces for immobilizing, respectively, can further be particles, membranes, matrices or solid phases.
The compounds of the present invention, preferably immobilized, are used for the fractionation of complex solutions or biological samples (e.g. bodily fluids, plasma, whole blood, serum, further samples derived from blood, cell suspensions, supernatants of cell cultures) and other biomolecule-containing solutions as well as for the purification of specific proteins (e.g. L-selectin, P-selectin, chemokines, coagulation factors) from these solutions/samples.
The dendritic polyglycerol sulfonates according to the present invention and/or the dendritic polyglycerol sulfates according to the present invention are used as capture molecules, e.g. in ELISA.

The dendritic polyglycerol sulfates and sulfonates have a great anti-inflammatory potential, because they combine the advantages of reported inhibitors:
easy synthesis (cost-effective)
biocompatibility (high similarity to heparan sulfate/heparin)
high affinity to L- and P-selectin (IC₅₀=10 or 40 nM, respectively, measurement in vitro). In vitro analysis in Biacore show, that activity (binding to L- and P-selectin) increases depending on their size.
binding of chemokins, which inhibit the activation of the leukocytes.

The dendritic polyglycerol sulfates of the invention are furthermore disclosed as inhibitors of leukocyte-endothelium interaction where L- and P-selectin ligand structure was simplified to sulfate groups and linked to a polyglycerol scaffold. The compounds were safely used and dampened immune response in a contact dermatitis mouse model. (See also Examples and Figures).

The present invention is illustrated by the following figures and examples.

### FIGURES

FIG. 1. Synthesis scheme of the dendritic polyglycerol sulfates.

FIG. 2. Synthesis scheme of the dendritic polyglycerol sulfonates.

FIG. 3. Inhibition of L-selectin ligand binding by selected dendritic polyglycerol sulfates.

Binding of L-selectin to its synthetic ligand (sLeX-tyrosine sulfate) was set at 100% (control value).

Average molecular weight of the polyglycerol core [g/mol]: 2a, 2500; 2c, 10,000; 2d, 20,000.

Degree of sulfation was about 80% for all polyglycerol sulfates (see also Table 1).

FIG. 4. Competitive inhibition of selectins by the dendritic polyglycerol sulfate 2c.

Binding to the synthetic ligand (sLeX-tyrosine sulfate) was set at 100% respectively (control value).

FIG. 5. Sulfation degree-dependent, competitive Inhibition of L-selectin ligand binding by derivatives of the dendritic polyglycerol 2d.

The derivatives were used with a concentration of 10 nM. Binding of L-selectin to its synthetic ligand (sLeX-tyrosine sulfate) was set at 100% (control value).

FIG. 6. Example of a dendritic polyglycerol sulphate (dPGS).

FIG. 7. dPGS do not Inhibit proliferation of the monocytic cell line THP-1.

Proliferation assay: Alamar Blue.

Vitality of T cells is not influenced by dPGS at different concentrations in comparison to prednisolone.

FIG. 8. dPGS show no induction of apoptosis on PBMCs.

A: PBMC+dPGS (+/−CD3 stimulus)

B: PBMC+dPGS (+/−LPS stimulus)

Apoptosis assay: Annexin V read-out.

FIG. 9. dPGS do not inhibit TNFα secretion in murine dendritic cells (DC).

Murine dendritic cells+dPGS (+/−LPS)

Assay: ELISA.

FIG. 10. dPGS show no interference of TNFα secretion in human T cells.

PBMC+/−anti CD3 Beads+dPGS

Assay: ELISA.

FIG. 11. Selectin binding specificity of dPGS.

The dPGS with a Mₙ of the polymeric core of 4.000 g/mol and a sulfation degree of 84% was used (dPGS 4000/85).

FIG. 12. Selectin binding depends on sulfation of the dendritic polyglycerol.

The dPGS with a Mₙ of the polymeric core of 6.000 g/mol was used (dPG 6000).

FIG. 13. Core size and sulfation rate dependent selectin binding of dPGS

FIG. 14. dPGS reduce edema formation in an acute TMA-induced allergic contact dermatitis model.

dPGS was injected into nuchal fold of mice.

FIG. 15. dPGS reduce granulocyte emigration after acute TMA-Induced allergic contact dermatitis.

dPGS was injected into nuchal fold of mice.

FIG. 16. dPGS reduce edema formation in a subchronic TMA-Induced allergic contact dermatitis model.

FIG. 17. dPGS prevent Infiltration of granulocytes and neutrophils in a subchronic TMA-Induced allergic contact dermatitis model.

A: granulocytes (peroxidase activity)

B: neutrophils (elastase activity), normalized to vehicle (=0)

FIG. 18. dPGS reduce IL-2 and IL-4 concentration at the site of inflammation.

Subchronic TMA challenge, ELISA of ear homogenates.

### EXAMPLES

### Example 1

### Synthesis of the Dendritic Polyglycerol Sulfates

The synthesis of the dendritic polyglycerol sulfates is carried out as substantially described in [11].

### Materials:

SO₃/pyridine complex was purchased from Fluka (Buchs, Switzerland). The reagent was used without further purification. The solvent N,N-dimethyl formamide (DMF, p.a. quality, purchased from Roth, Karlsruhe, Germany) was dried over CaH₂ and stored over molecular sieve 4 Å prior to further use. Dialysis was carried out with tubing of regenerated cellulose (SpectraPore 6/Roth) in distilled water in a 5 l beaker, wherein the solvent was changed three times over a period of 48 hours.

### 1. Polymeric Polyglycerol Corer

Polyglycerol 1 is a readily available, well defined polymer with dendritic (tree-like) branching, which is obtained by controlled anionic polymerization of glycidol [12-14]. The degree of branching of 1 (60%) is lower than that of a perfect glycerol dendrimer (100%) [15]. However, the physico-chemical characteristics are similar [16]. The molecular weight (1,000-30,000 g/mol) and the degree of polymerization (DP) can readily be tailored via the ratio of monomer and initiator and narrow polydispersities are obtained (typically <2.0) [14]. Due to the biocompatible characteristics of the aliphatic polyether polyol (e.g. polysaccharides, poly(ethyleneglycol)s) in general similar characteristics are anticipated of polyglycerol [13]. In addition, oligoglycerols (with 2-10 monomer units) were studied in detail with respect to their toxicological characteristics and were approved as nutritional and pharmacological additives [16,17]. Thus, the dendritic polyglycerol 1 should be suitable as a highly functional carrier for drugs, and for the generation of dendritic polyanions (polysulfates, polycarboxylates, polysulfonates).

Furthermore, the polyglycerols (PG) 1a (Mₙ=2,500 g/mol, M_{w}/Mₙ=1.6), 1b (Mₙ=5,000 g/mol, M_{w}/Mₙ=1.6), 1c (Mₙ=10,000 g/mol, M_{w}/Mₙ=1.8) and 1d (Mₙ=20,000 g/mol, M_{w}/Mₙ<2.0) were prepared using 1,1,1-tris(hydroxymethyl)propane (TMP) as initiator in case of 1a-c and pentaerythrol (PE) as initiator in case of 1d, as previously described [14].

### 2. Analysis

¹H NMR and ¹³C NMR spectra were recorded in D₂O at concentrations of 100 mg/ml in a Broker ARX 300 spectrometer, which operates at 300 or 75.4 MH, respectively. IR measurements were performed at a Bruker IFS 88 FT-IR spectrometer using a KBr plate. The degree of sulfation (ds) (Table 1) of compounds 2a-d was determined using elemental analysis.

### 3. Synthesis of the Polyglycerol Sulfates

The synthesis of the polyglycerol sulfates was carried out by modifying an established method for the sulfation of β-1,3-glukanes which was described by Alban et al. [18], using dendritic polyglycerols with different molecular weights (1a-d) as core polymers and the SO₃/pyridine complex as sulfation reagent in dry DMF as solvent (see Scheme 1). The concentration of the SO₃/pyridine complex in DMF as well as its molar ratio (SO₃ per OH groups) was kept constant in all cases.

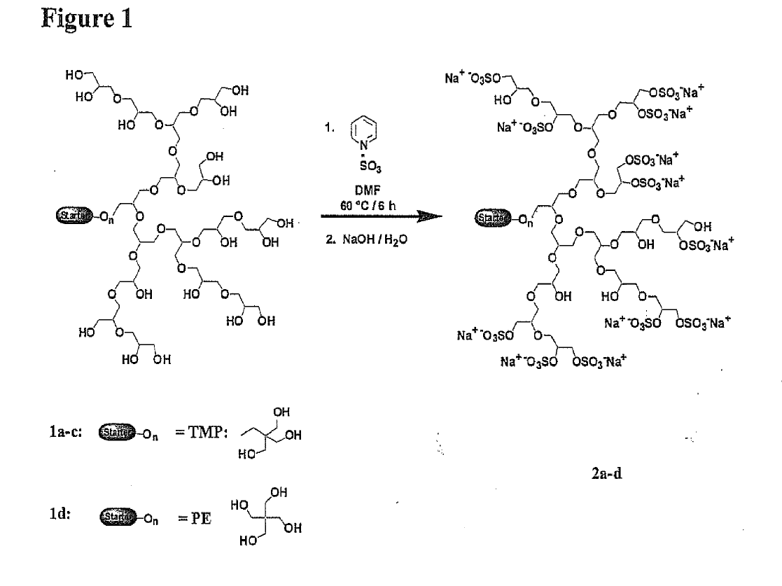

For a synthesis scheme see FIG. 1.

To a stirred solution of 5.0 g polyglycerol (1a, 1b, 1c, 1d) (67.5 mmol OH groups) in 25 ml DMF a solution of 10.75 g (67.5 mmol) SO₃/pyridine complex in 67.5 ml DMF was added drop-wise for 4 hours at 60° C. under an argon atmosphere. After stirring the reaction mixture for additional 2 hours at 60° C. and 18 hours at room temperature, 50 ml distilled water were added. To the aqueous solution immediately 1 M NaOH were added until reaching a pH of 11. Concentration in vacuum resulted in the raw product, which was further purified by dialysis in water. After evaporating the solvent polyglycerol sulfates 2a-d were obtained as light yellow solids, which were further dried over P₂O₂.

The polyglycerol sulfates (2a-d) were obtained in good yields (50-75%) and high purities (>98% according to ¹H NMR) after dialysis in distilled water.

Yields: 7.49 g (2a); 8.96 g (2b); 7.01 g (2c); 6.86 g (2d).

¹H NMR (300 MHz, D₂O): δ (ppm) 0.98 [t, 3H, CH₃CH₂C(CH₂O)₃—PG-OSO₃Na], 1.48 [m, 2H, CH₃CH₂C—(CH₂O)₃—P—OSO₃Na], 3.40-4.00 [m, CH₃CH₂C(CHO)₃—PG-OSO₃Na], 4.19, 4.33, 4.38 [PG-OSO₃Na], 4.72 [PG-OCH₂CH(OSO₃Na)CH₂OSO₃Na].

Note: in case of 2d the peaks at 0.98 and 1.48 do not apply.

¹³C NMR (D₂O, 75.4 MHz): δ (ppm) 66.9, 67.6, 68.2, 69.4, 70.3, 75.8, 77.2, 78.3 [PG-OSO₃Na].

IR (KBr): v (cm⁻¹) 3470 [OH], 2930 [CH], 1260 [S═O], 780 [C—O—S].

Sulfur content after elemental analysis: 2a: 15.38% S, 2b: 14.28% S, 2c: 15.20% S, 2d: 13, 96%.

By ¹H-NMR spectroscopy no degradation of the polyglycerol core was observed.

Using a SO₃/pyridine concentration which was equimolar to the OH groups about 85% of all free OH groups were sulfated (Table 1). This high degree of sulfation shows that polyglycerols are more easily accessible to sulfation than polysaccharides (24).

TABLE 1
Characterization of the dendritic polyglycerol sulfates 2a-c

| | | | Mₙ | | Mₙ |
|---|---|---|---|---|---|
| | | | of the | | of the |
| | | | polymer | Degree of | polyglycerol |
| Polyglycerol | Polymer | | core^{a} | sulfation^{b} | derivative^{c} |
| derivative | core | DPₙ | [g/mol] | [%] | [g/mol] |
| Sulfate 2a | 1a | 32 | 2,500 | 85 | 5,500 |
| Sulfate 2b | 1b | 66 | 5,000 | 79 | 10,500 |
| Sulfate 2c | 1c | 133 | 10,000 | 84 | 21,700 |
| Sulfate 2d | 1d | 269 | 20,000 | 76 | 40,900 |

^{a}determined by NMR and/od GPC (DMF).
^{b}degree of sulfation (ds) obtained by elemental analysis;
^{c}calculated using Mₙ of the polymer core and the experimental measure of functionalization.
Mₙ = average molecular weight of the polyglycerol core;
DPₙ = degree of polymerization of the polyglycerol core;

The detailed analysis of all starting materials 1a-d and products 2a-d by NMR, IR and elemental analysis confirmed the structure and the degree of functionalization of these dendritic polyglycerol sulfates. The molecular weights of the non-functionalized polyglycerols were determined by using ¹H NMR data after precipitation.

### Example 2

### Synthesis of the Dendritic Polyglycerol Sulfonates

### Materials

The sodium salt of vinylsulfonic acid (25% solution by weight in water) was commercially obtained from the company Sigma-Aldrich and used without further purification. For the dialysis of the synthesized sulfonates in water dialysis tubing made of regenerated cellulose from the company Roth (SpectraPor6) with a MWCO of 1,000 g/mol vas used.

### 1. Polymeric Polyglycerol Cores

See Example 1.

### 2. Analytics

NMR spectroscopy: ¹H-NMR and ¹³C-NMR spectra were recorded with a Bruker ARX 300 spectrometer at 300 MHz or 75.4 MHz, respectively, in D₂O at concentrations of 100 mg/ml. The degree of sulfonation was determined using elemental analysis.

### 3. Synthesis of the Polyglycerol Sulfonates

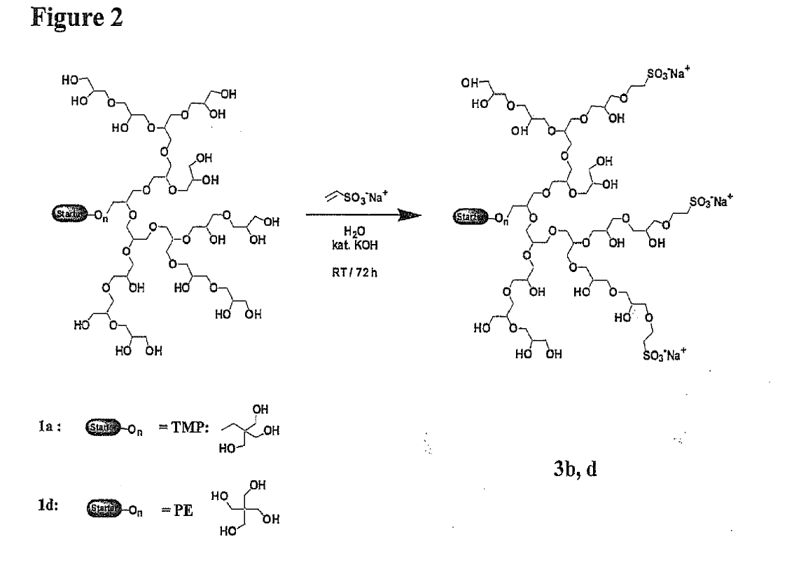

For a synthesis scheme see FIG. 2.

10 g polyglycerol 1b, 1d (2.0 mmol; approx. 135 mmol OH groups) were dissolved in 20 ml water and a solution of 757 mg (13.5 mmol) potassium hydroxide in 1 ml water were added reaching a 10% deprotonation of the OH groups of the polyglycerol. The reaction solution was cooled to approx. 5° C. with the aid of an ice bath. Then, sodium salt of vinylsulfonic acid (26.347 g; 202.5 mmol) in form of a 25% by weight, aqueous solution were slowly added for 4 hours via a dropping funnel. After the addition was completed the reaction mixture was heated to RT and stirred for another 3 days. After removing the solvent in vacuum, the obtained raw product was further purified by dialyzing in water for 24 hours, wherein the water was changed three times. Afterwards the raw product was concentrated in vacuum and dried for removing the remaining water in an exsiccator over phosphor pentoxide. The synthesized polyglycerol sulfates 3b, 3d were obtained in form of a slightly yellow colored highly viscous liquid with a degree of functionalization of 3 to 10%.

Yield: 3b 6.58 g, 3d: 5.48 g.

¹H-NMR (D₂O, 300 MHz): δ (ppm)=0.88 [t, 3H, CH₃CH₂C(CH₂O)-PG-CH₂CH₂SO₃Na], 1.42 [m, 2H, CH₃CH₂C(CH₂O)₃-PG-CH₂CH₂SO₃Na], 3.21 [t, 2H, CH₃CH₂C(CH₂O)₃-PG-CH₂CH₂SO₃Na]3.35-4.05 [m, CH₃CH₂C(CH₂O)₃-PG-CH₂CH₂SO₃Na];

¹³C-NMR (D₂O, 75.4 MHz): δ (ppm)=53.0 [PG-CH₂CH₂SO₃Na], 63.3, 65.1 [PG-CH₂CH₂SO₃Na], 68.2 [PG-CH₂CH₂SO₃Na], 71.4, 72.9, 74.7, 80.5, 82.0 [PG-CH₂CH₂SO₃Na].

Results of the elemental analysis: 3b: 0.58% S, 3d: 1.30% S.

By ¹H-NMR spectroscopy no degradation of the polyglycerol core was observed.

TABLE 2
Characterization of the dendritic polygylcerol sulfonates 3b and 3d

| | Mₙ | Mₙ (theoret.) | Degree of | |
|---|---|---|---|---|
| Polyglycerol | of the PG core | at f = 100% | sulfonation^{b} | Mₙ (exp.) |
| sulfonate | [g/mol] | [g/mol] | [%] | [g/mol] |
| 3b | 5,000 | 13,900 | 4 | 5,200 |
| 3d | 20,000 | 55,300 | 8 | 21,800 |

^{b}degree of sulfonation, obtained by elemental analysis;
Mₙ = average molecular weight of the polyglycerol core;

### Example 3

### Binding of the Dendritic Polyglycerol Sulfates to Selectin In Vitro

In a competitive binding assay the binding of the polyglycerol sulfates to L-, P- and E-selectin was analyzed by surface plasmon resonance in Biacore X. In this approach the selectins are at first immobilized on colloidal gold beads. Then, the binding of the analyte to the selectin ligand sLeX-tyrosine sulfate which is coupled to the sensor chip is measured. By preincubating the analyte with the polyglycerol sulfates the binding of the analyte to the chip-coupled ligand is decreased in a concentration-dependent manner when the interaction of the polyglycerol sulfates with the binding domain of the ligand of the selectins is specific. In this case a decrease of the binding signal is observed.

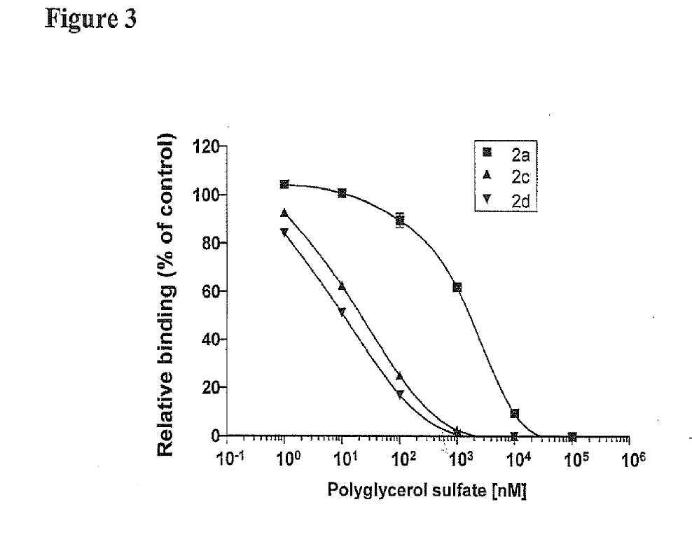

FIG. 3 shows the concentration-dependent inhibition of L-selectin ligand binding by selected polyglycerol sulfates. With increasing molecular weight the polyglycerol sulfates show an increasing inhibitory potential with a comparable degree of sulfation. As apparent from FIG. 3, compound 2d has an IC₅₀ value of about 10 nM.

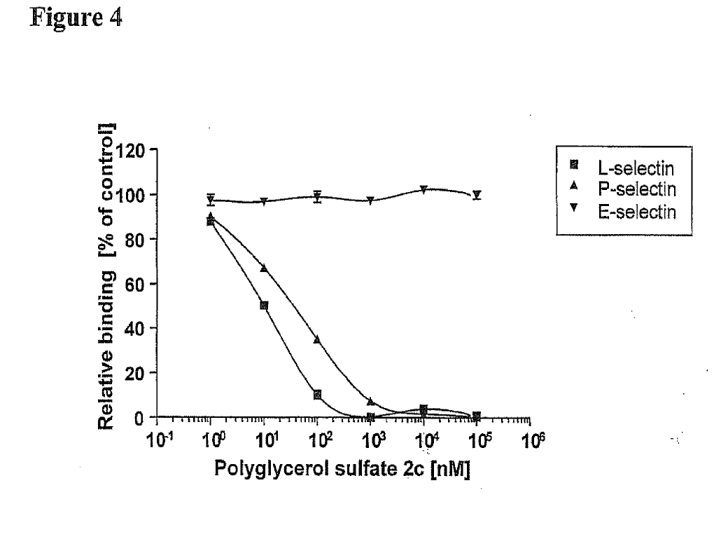

For a further characterization of selectin-specific binding inhibition curves of L-, P- and E-selectin after preincubation with the polyglycerol derivative 2c were obtained (see FIG. 4). Here it appears, that L-selectin is inhibited best by the derivative 2c (IC₅₀=10 nM), for P-selectin the compound has an IC₅₀ of 30 nM, whereas E-selectin is not inhibited.

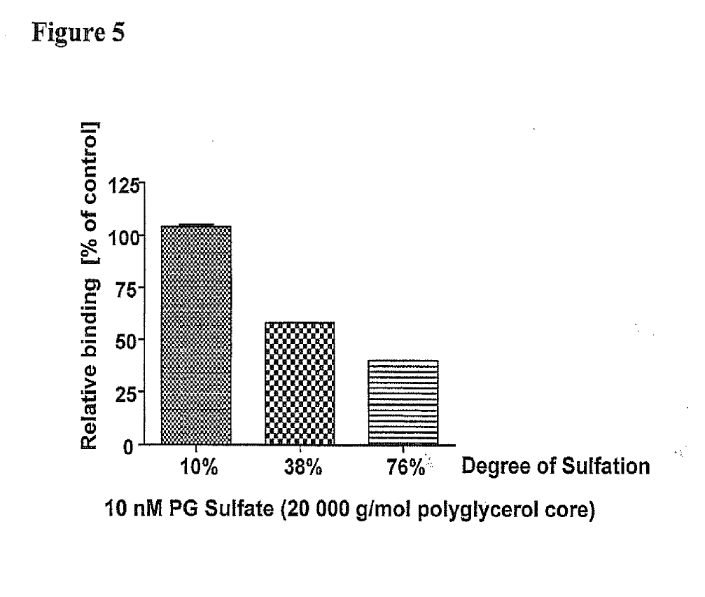

The influence of the degree of sulfation of the dendritic polyglycerols on the L-selectin binding was investigated for the example of derivative 2d (Mₙ of the PG core=20,000 [g/mol]). The derivative 2d was used with a concentration of 10 nM and sulfation degrees of 10%, 38% and 76%. Again, the influence of the polyglycerol sulfates on the interaction between the analyte L-selectin and the immobilized ligand sLeX-tyrosine sulfate was measured (competitive binding assay, see above). The control value was set at 100%, which corresponds to the binding signal which is generated by the interaction between L-selectin and the chip-coupled ligand sLeX-tyrosine. By preincubating the analyte L-selectin with 10 nM of the differently sulfated polyglycerol derivatives a reduction of the L-selectin-binding signal is measured with an increasing degree of sulfation, which is shown in FIG. 5 as percental value compared to the control value. The 10% sulfation of 2d obviously appears to be not sufficient to interact with L-selectin during the preincubation phase; the binding signal corresponds to the control value. The 38% sulfation of 2d reduces the L-selectin ligand binding to about 60% of the binding signal of the control value and the 76% sulfation of 2d reduces it to about 45% of the control value. These measurements show that the degree of sulfation and binding affinity correlate positively. Furthermore, a particularly threshold value of the sulfation degree appears to be necessary in order to accomplish an interaction with L-selectin.

### Example 4

### Dendritic Polyglycerol (dPG) and Sulfated Derivatives (dPGS)

Dendritic polyglycerols are well defined polymers with treelike branching. The detailed synthesis was carried out as described in Example 1. The degree of polymerisation and branching can easily be tailored and narrow polydispersities can be obtained.

We synthesized different core structures with molecular weights (MW) between 240 and 6,000 Da, as described in Example 1. The compounds were further functionalized with the SO₃/pyridine complex as sulfation reagent. The percentage loading of sulfate (degree of sulfation) on the dendritic polyglycerol scaffold was determined by elemental analysis and ranged from 10-92%.

dPG and dPGS were stored at 4° C., aqueous solutions were stable after 6 month storage at −20° C.

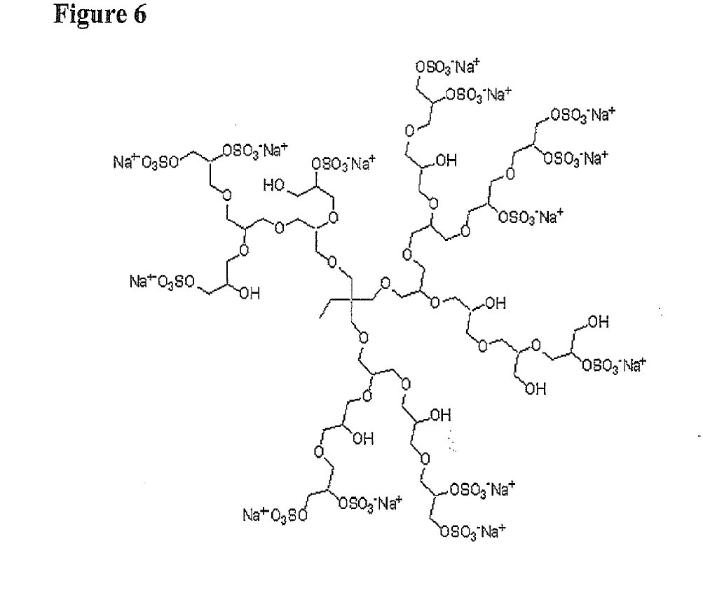

For an example of a dPGS see FIG. 6.

TABLE 3
Characterization of the dendritic polyglycerol sulfates (dPGS)

| | Mₙ of the | | Mₙ of the | |
|---|---|---|---|---|
| | polyglycerol | Degree of | polyglycerol | Previous name |
| | core | sulfation | derivative | of the |
| Derivative | [g/mol] | [%] | [g/mol] | derivative |
| dPG | 3,000 | 0 | 3,000 | |
| dPGS2500/85 | 2,500 | 85 | 5,500 | 2a |
| dPGS2500/92 | 2,500 | 92 | 6,800 | |
| dPGS4000/84 | 4,000* | 84 | 8,600 | 2c |
| dPGS6000/76 | 6,000* | 76 | 12,300 | 2d |

dPG = dendritic polyglycerol
dPGS = dendritic polyglycerol sulfate
*molecular weights have been re-determined

### Example 5

### Cytotoxicity and Immuno-Regulating Properties of Polyglycerol Sulfates

To test whether the polyanionic dPGS of the invention could be used safely in cell culture and in vivo in mice the compound dPGS6000/76 was characterized exemplarily in detail.

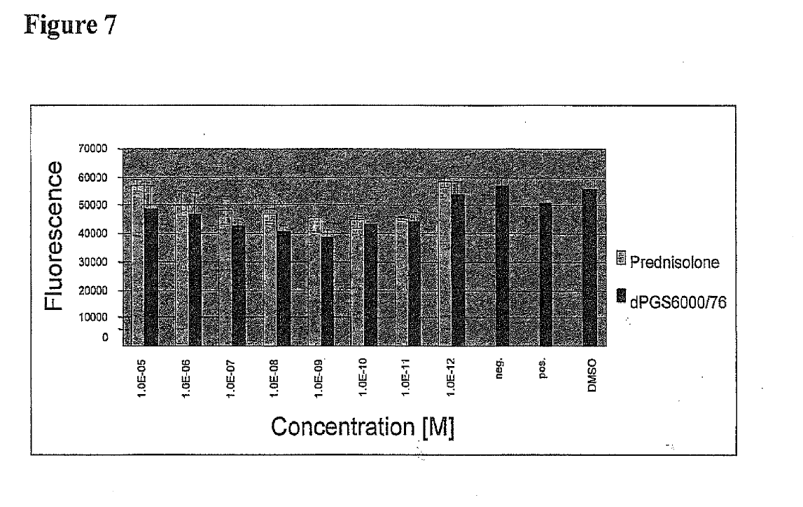

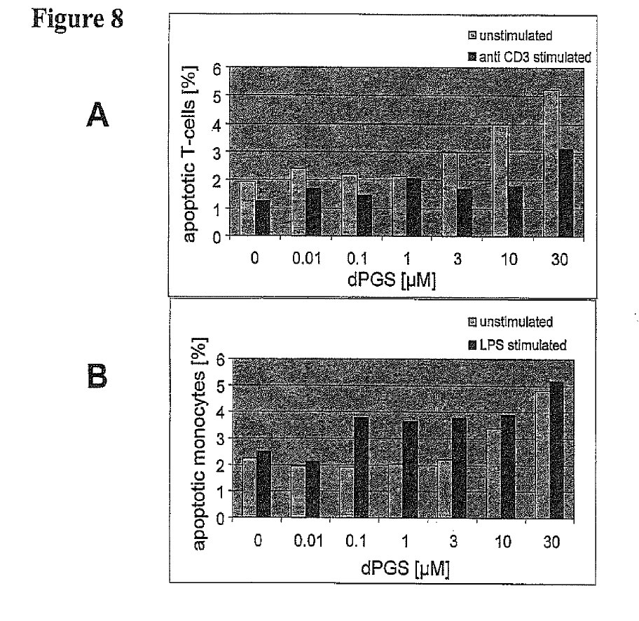

To test cellular toxicity, we performed proliferation assays with the monocytic cell line THP-1. The compound dPGS6000/76 showed no inhibition of cellular proliferation up to a concentration of 10 μM (see FIG. 7). When peripheral blood mononuclear cells (PBMCs) were cultured in the presence of up to 30 μM dPGS6000/76 for 24 h only a slight increase of apoptotic cells was observed irrespective of cellular stimulation (see FIG. 8).

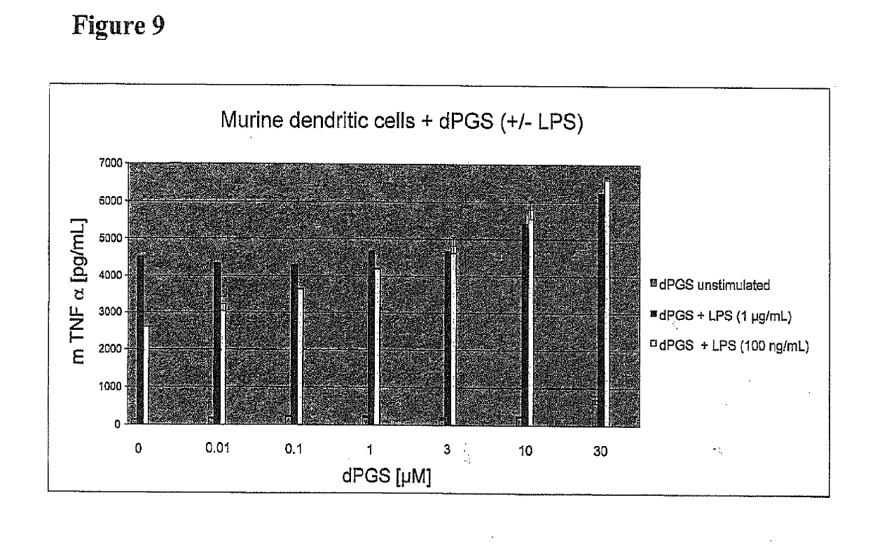

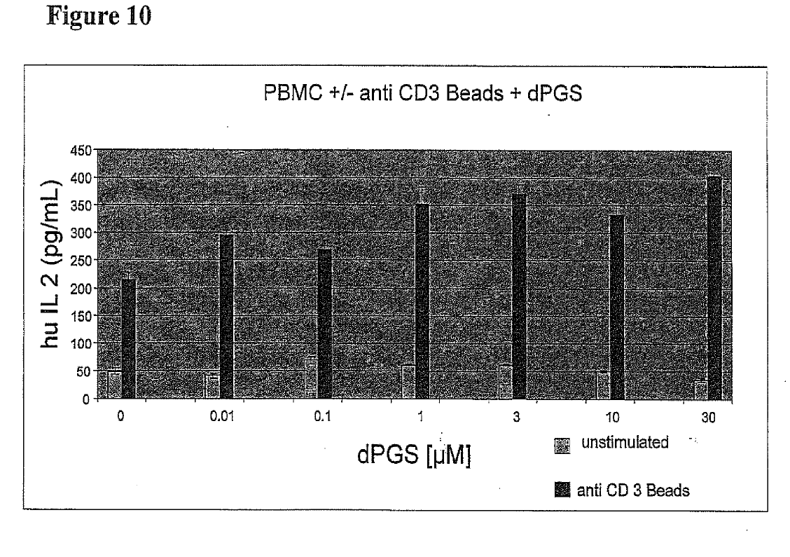

We next examined the influence of dPGS on cellular immuno-regulating activity. Cytokine release was characterized on murine dendritic cells (see FIG. 9) and the T cell fraction of human PBMCs (see FIG. 10). Compared to the control (no dPGS added) the concentration of m TNFa and hu IL-2 did not change significantly.

### Example 6

### Dendritic Polyglycerol Sulfates Block Selectin-Ligand Binding In Vitro

To evaluate selectin-binding of dPGS In vitro we applied a highly sensitive Biacore-based competitive binding assay, as described in Example 3, which allows to determine 50% inhibitory concentrations (IC₅₀) of inhibitory compounds.

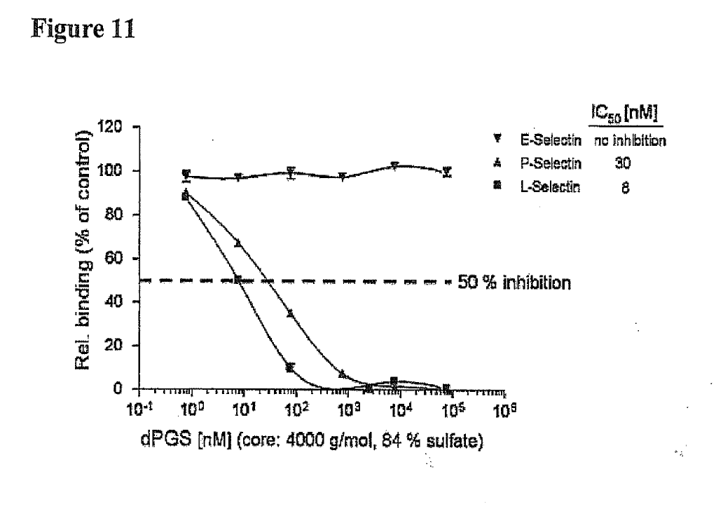

The selectin specificity of dPGS was analyzed. Whereas E-selectin binding was not affected by dPGS4000/84, L- and P-selectin were inhibited efficiently and gave IC₅₀ values of 8 and 30 nM (see FIG. 11). (These experiments were carried out as described in Example 3 and for reconfirmation.)

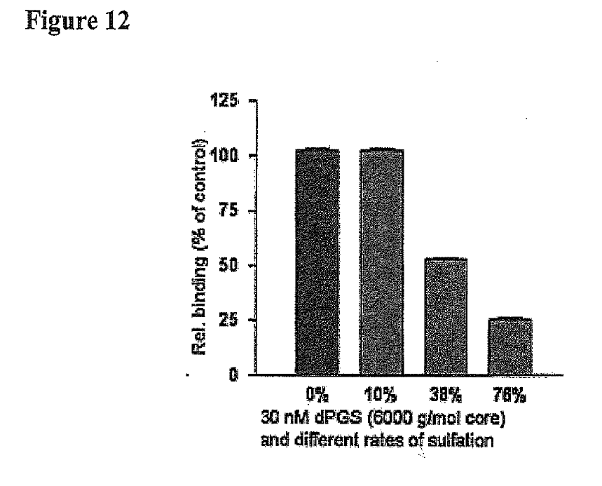

Sulfate dependency of selectin binding was then confirmed with compounds bearing a different functionalization on the same scaffold. At a defined concentration of 30 nM the inhibitory effect of the derivatives was studied (see FIG. 12). Core structure dPG6000 with no or 10% sulfate did not interfere with L-selectin-ligand binding, in contrast 38% and 76% sulfation reduced the relative binding to 55% or 26%, respectively. (These experiments were carried out as described in Example 3 and for reconfirmation.)

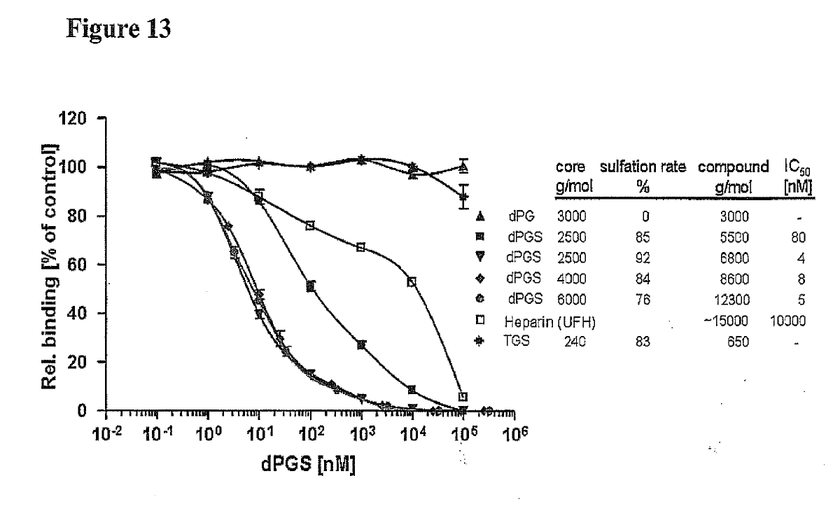

The influence of the dendrimer core size on selectin inhibition was then characterized (see FIG. 13). Dendritic polyglycerols with molecular weights ranging from 240 Da (3 monomer units) to 6,000 Da (80 monomer units) were synthesized and further highly sulfated. The degree of functionalization was in the range from 76 to 92%.

The small compound triglycerol (TGS) 240/83 showed no inhibition on L-selectin binding up to the high micromolar range but for compound dPGS2500/85 the IC₅₀ was 80 nM. By increasing the degree of sulfation another 7% the IC₅₀ value of the resulting compound dPGS2500/92 further decreased to 4 nM.

It is obvious that selectin binding requires a critical size of the polymer core but density of sulfate groups (degree of sulfation) on the polymeric scaffold seems to be of even greater importance. Further increase of the core structure and equal functionalization did not improve selectin binding. For comparison the L- and P-selectin binding polymer heparin was included to this study. This polysulfated glucosaminoglycan has an average molecular weight of 15,000 Da and carries about 2.4 sulfates per disaccharide. The IC₅₀ value on L-selectin binding of this compound was 15 μM and hence about 4000 fold greater than dPGS2500/92.

TABLE 4
Core size and sulfation rate dependent selectin binding of dPGS.

| | Mₙ of the | | Mₙ of the | |
|---|---|---|---|---|
| | polyglycerol | Degree of | polyglycerol | |
| | core | sulfation | derivative | IC₅₀ |
| Derivative | [g/mol] | [%] | [g/mol] | [nM] |
| dPG | 3,000 | 0 | 3,000 | no inhibition |
| dPGS2500/85 | 2,500 | 85 | 5,500 | 80 |
| dPGS2500/92 | 2,500 | 92 | 6,800 | 4 |
| dPGS4000/84 | 4,000 | 84 | 8,600 | 8 |
| dPGS6000/76 | 6,000 | 76 | 12,300 | 5 |
| Heparin (UFH) | n.d. | n.d. | ~15,000 | 10,000 |
| TGS | 240 | 83 | 650 | no inhibition |

dPG = dendritic polyglycerol
dPGS = dendritic polyglycerol sulfate
TGS = triglycerol
n.d. not determined

### Example 7

### dPGS Reduce Leukocyte Recruitment in Acute and Subchronic Skin Inflammation Model

We then investigated the influence of the dendritic polyglycerol sulfates in a murine model for skin inflammation.

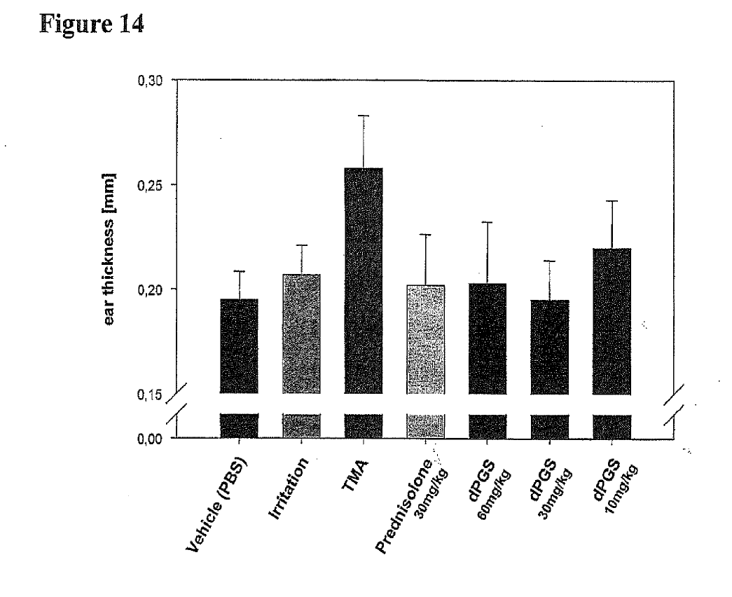

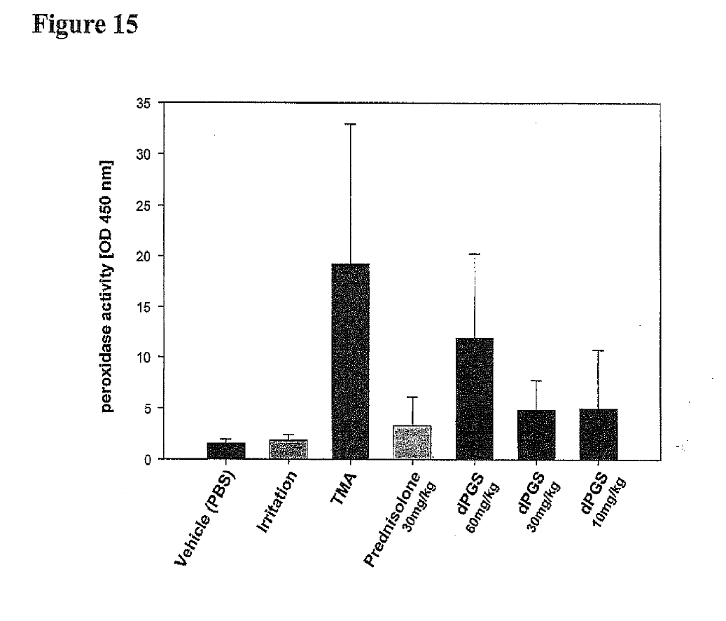

In an acute TMA-induced inflammatory response the compound dPGS6000/76 prevented edema formation and therefore ear swelling. At a dose of 30 mg/kg the antiinflammatory efficacy was comparable the corticosteroid prednisolone (see FIG. 14). This antiinflammatory effect was attributed to the dPGS-mediated reduction of granulocyte emigration (see FIG. 15).

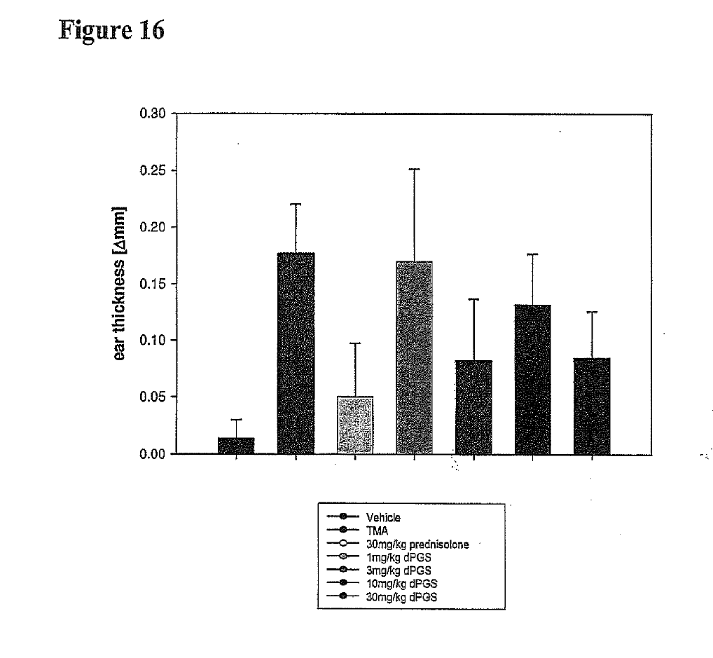

In a subchronic inflammation model 8 days after TMA challenge the protecting effect of dPGS was still obvious. Ear thickness of dPGS treated mice was reduced but not as effective as in the prednisolone positive control (see FIG. 16).

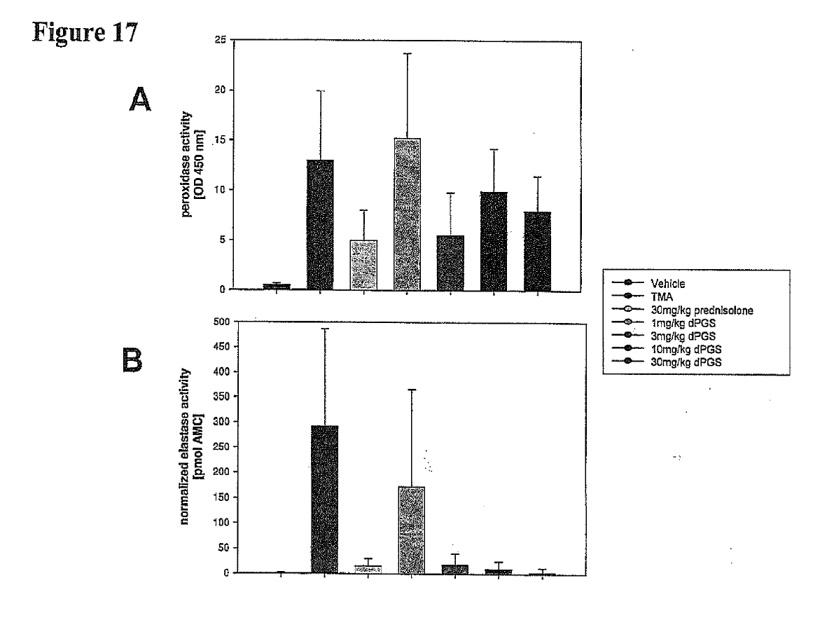

In addition still a clear reduction in granulocyte and neutrophil infiltration was measured (see FIG. 17) and comparable to the prednisolone standard.

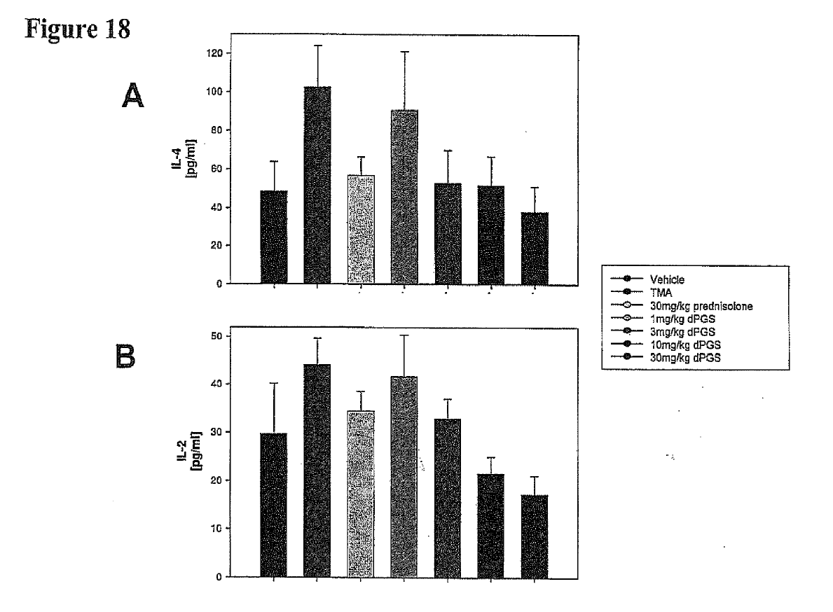

We then analysed activation of naïve T cells by measuring cytokine levels in mice ear homogenates. The obvious concentration-dependent decrease of Th1-type IL-2 and the Th2-type IL-4 in dPGS treated mice further indicates that dPGS damp down the T cell dependent skin inflammation in TMA-induced contact hypersensitivity (see FIG. 18).

### REFERENCES

1. Ley, K. (2003) The role of selectins in inflammation and disease. Trends Mol Med., 9(6): 263 8.
2. Springer, T. A. (1990) Adhesion receptors of the immune system. Nature, 346: 425-434.
3. Lefer, D. F. (2000) Annu Rev. Pharmacol. Toxicol., 40: 283-294.
4. Boehncke, W H et al. (2005) Exp. Dermatol., 14(1): 70-80.
5. Simanek et al., (1998) Selectin-carbohydrate interactions: From natural ligands to designed mimics. Chem. Rev., 98(2): 833-862.
6. Boehncke W H et al., (2006) Biologic therapies for psoriasis. A systematic review. J. Rheumatol., 33: 1447-1451.
7. Willburger et al., (2006) Zertifizierte medizinische Fortbildung: Pharmakologische Therapie der rheumatoiden Arthritis. Dtsch Arstebl; 103(1-2): A 48-57
8. Ulbrich H., et al. (2003) Leukocyte and endothelial cell adhesion molecules as targets for therapeutic interventions in inflammatory disease. Trends Pharmacol Sci., 12: 640-647.
9. Mowery, P et al. (2004) Synthetic glycoprotein mimics inhibit L-selectin-mediated rolling and promote L-selectin shedding. Chem. Biol. 11: 752-732.
10. Rele, S M et al. (2005) Dendrimer-like PEO glycopolymers exhibit anti-inflammatory properties. J. Am. Chem. Soc., 127: 10132-10133.
11. Türk, H.; Haag, R. and Alban, S. (2004) Dendritic Polyglycerol Sulfates as New Heparin Analogues and Potent Inhibitors of the Complement System. Bioconjugate Chem. 15: 162-167.
12. Sunder, A., Mülhaupt, R., Haag, R., and Frey, H. (2000) Hyperbranched Polyether Polyols: A Modular Approach to Complex Polymer Architectures. Adv. Mater. 12, 235-239.
13. Frey, H., and Haag, R. (2002) Dendritic polyglycerol: a new versatile biocompatible material. Rev. Mol. Biotech. 90: 257-267.
14. Sunder, A., Hanselmann, R., Frey, H., and Mülhaupt, R. (1999) Controlled Synthesis of Hyperbranched Polyglycerols by Ring-Opening Multibranching Polymerization. Macromolecules 32: 4240-4246.
15. Haag, R., Sunder, A., and Stumbé, J.-F. (2000) An Approach to Glycerol Dendrimers and Pseudo-Dendritic Polyglycerols. J. Am. Chem. Soc. 122, 2954-2955.
16. Wilson, R., Van Schie, B J., and Howes, D. (1998) Overview of the Preparation, Use and Biological Studies on Polyglycerol Polyricinoleate (PGPR). Food Chem. Toxicol. 36: 711-718.
17. Howes, D., Wilson, R., and James, C. T. (1998) The Fate of Ingested Glyceran Esters of Condensed Castor Oil Fatty Acids [Polyglycerol, Polyricinoleate (PGPR)] in Rat. Food Chem. Toxicol. 36: 719-738.
18. Alban, S., Kraus, J., and Franz, G. (1992) Synthesis of Laminarin Sulfates with Anticoagulant Activity. Arzneim.-Forsch./Drug Res. 42:1005-1008.

1-46. (canceled)

47. Dendritic polyglycerol sulfonate, characterized by
a) a polymeric polyglycerol core, composed of repeated units of glycerin with the formula (RO—CH₂)₂CH—OR
on a multifunctional starter molecule, which is a polyhydroxy compound having 1 to 1,000 OH groups,
wherein R═H or further glycerin units,
the core having a branching degree of 0 to 100% and
an average molecular weight of 100 to 1,000,000 g/mol,
b) the substitution of one or more OH groups of the glycerin units with —SO₃H or —SO₃Na groups
or the attachment of an oligomeric spacer at one or more OH groups of the glycerin units,
the oligomeric spacer having the generic formula
—(CH₂)ₙ— or —[(CH₂)ₘ—O)]ₙ—, wherein m is 1 to 100 and n is 1 to 50,000, and bound thereto —SO₃H or —SO₃Na groups,
so that a degree of sulfonation of 1 to 100% is obtained,
and
c) a molecular weight of 110 to 1,500,000 g/mol;
or
a dendritic polyglycerol sulphate loaded with signalling molecules or having signalling molecules bound thereto, wherein the dendritic polyglycerol sulphate is characterized by:
a) a polymeric polyglycerol core, composed of repeated units of glycerin with the formula (RO—CH₂)₂CH—OR
on a multifunctional starter molecule, which is a polyhydroxy compound having 1 to 1,000 OH groups,
wherein R═H or further glycerin units,
the core having a branching degree of 0 to 100% and
an average molecular weight of 100 to 1,000,000 g/mol,
b) the substitution of one or more OH groups of the glycerin units with —OSO₃H or —OSO₃Na groups
or the attachment of an oligomeric spacer at one or more OH groups of the glycerin units,
the oligomeric spacer having the generic formula
—(CH₂)ₙ— or —[(CH₂)ₘ—O)]ₙ—, wherein m is 1 to 100 and n is 1 to 50,000, and bound thereto —OSO₃H or —OSO₃Na groups,
so that a degree of sulfation of 1 to 100% is obtained,
and
c) a molecular weight of 200 to 5,000,000 g/mol.

48. Method for producing a dendritic polyglycerol sulfonate compound according to claim 47, comprising the use of a multifunctional starter molecule and a sulfonation reagent.

49. A method for inhibiting selectin, comprising bringing a compound of claim 47 together with selectin.

50. A method for binding a protein, comprising bringing a compound of claim 47 together with a protein.

51. A method for the treatment of an inflammatory disease, comprising
A) administering an effective amount of a dendritic polyglycerol sulfonate to a subject in need thereof, which has the following characteristics
a) a polymeric polyglycerol core, composed of repeated units of glycerin with the formula (RO—CH₂)₂CH—OR
on a multifunctional starter molecule, which is a polyhydroxy compound having 1 to 1,000 OH groups,
wherein R═H or further glycerin units,
the core having a branching degree of 0 to 100% and
an average molecular weight of 100 to 1,000,000 g/mol,
b) the substitution of one or more OH groups of the glycerin units with —SO₃H or —SO₃Na groups
or the attachment of an oligomeric spacer at one or more OH groups of the glycerin units,
the oligomeric spacer having the generic formula
—(CH₂)ₙ— or —[(CH₂)ₘ—O)]ₙ—, wherein m is 1 to 100 and n is 1 to 50,000, and bound thereto —SO₃H or —SO₃Na groups,
so that a degree of sulfonation of 1 to 100% is obtained,
and
c) a molecular weight of 110 to 1,500,000 g/mol;
or
B) administering to a subject in need thereof an effective amount of a dendritic polyglycerol sulfate, that has the following characteristics
a) a polymeric polyglycerol core, composed of repeated units of glycerin with the formula (RO—CH₂)₂CH—OR
on a multifunctional starter molecule, which is a polyhydroxy compound having 1 to 1,000 OH groups,
wherein R═H or further glycerin units,
the core having a branching degree of 0 to 100% and
an average molecular weight of 100 to 1,000,000 g/mol,
b) the substitution of one or more OH groups of the glycerin units with —OSO₃H or —OSO₃Na groups
or the attachment of an oligomeric spacer at one or more OH groups of the glycerin units,
the oligomeric spacer having the generic formula
—(CH₂)ₙ— or —[(CH₂)ₘ—O)]ₙ—, wherein m is 1 to 100 and n is 1 to 50,000, and bound thereto —OSO₃H or —OSO₃Na groups,
so that a degree of sulfation of 1 to 100% is obtained,
and
c) a molecular weight of 200 to 5,000,000 g/mol.

52. A method according to claim 51, wherein the inflammatory disease is an ischemia reperfusion damage or graft repulsion.

53. A method according to claim 51, wherein the dendritic polyglycerol sulfonate has the following characteristics
a) a polymeric polyglycerol core built on a multifunctional starter molecule having 1 to 4 OH groups.

54. A method according to claim 51, wherein the dendritic polyglycerol sulfonate has the following characteristics
a) a polymeric polyglycerol core built on a multifunctional starter molecule, which contains one or more further heterofunctionalities.

55. A method according to claim 51, wherein the dendritic polyglycerol sulfonate has the following characteristics
a) a polymeric polyglycerol core having a branching degree of 60%.

56. A method according to claim 51, wherein the dendritic polyglycerol sulfonate has the following characteristics
a) a polymeric polyglycerol core having an average molecular weight of 1,000 to 20,000 g/mol.

57. A method according to claim 51, wherein the dendritic polyglycerol sulfonate has the following characteristics
b) a degree of sulfonation of 30%.

58. A method according to claim 51, wherein the dendritic polyglycerol sulfonate has the following characteristics
b) a degree of sulfonation of 30% to 100%.

59. A method according to claim 51, wherein the dendritic polyglycerol sulfonate has the following characteristics
c) a molecular weight of 1,100 to 30,000 g/mol.

60. A method according to claim 51, wherein the dendritic polyglycerol sulfonate is loaded with signalling molecules or has signalling molecules bound thereto.

61. A method according to claim 60, wherein the signalling molecules are selected from the group consisting of radioactively labelled derivatives, dyes, fluorophores and chromophores.

62. A method according to claim 51, wherein the dendritic polyglycerol sulfonate is immobilized to a matrix.

63. A method according to claim 62, wherein the matrix is of inorganic or polymeric nature.

64. A method for inhibiting selectin, comprising bringing a dendritic polyglycerol sulfate together with selectin, wherein the dendritic polyglycerol sulfate is characterized by:
a) a polymeric polyglycerol core, composed of repeated units of glycerin with the formula (RO—CH₂)₂CH—OR
on a multifunctional starter molecule, which is a polyhydroxy compound having 1 to 1,000 OH groups,
wherein R═H or further glycerin units,
the core having a branching degree of 0 to 100% and
an average molecular weight of 100 to 1,000,000 g/mol,
b) the substitution of one or more OH groups of the glycerin units with —OSO₃H or —OSO₃Na groups
or the attachment of an oligomeric spacer at one or more OH groups of the glycerin units,
the oligomeric spacer having the generic formula
—(CH₂)ₙ— or —[(CH₂)ₘ—O)]ₙ—, wherein m is 1 to 100 and n is 1 to 50,000, and bound thereto —OSO₃H or —OSO₃Na groups,
so that a degree of sulfation of 1 to 100% is obtained,
and
c) a molecular weight of 200 to 5,000,000 g/mol;
or
a method for indicating selectin, comprising bringing a dendritic polyglycerol sulfate together with selectin, wherein the dendritic polyglycerol sulfate is characterized by:
a) a polymeric polyglycerol core, composed of repeated units of glycerin with the formula (RO—CH₂)₂CH—OR
on a multifunctional starter molecule, which is a polyhydroxy compound having 1 to 1,000 OH groups,
wherein R═H or further glycerin units,
the core having a branching degree of 0 to 100% and
an average molecular weight of 100 to 1,000,000 g/mol,
b) the substitution of one or more OH groups of the glycerin units with —OSO₃H or —OSO₃Na groups
or the attachment of an oligomeric spacer at one or more OH groups of the glycerin units,
the oligomeric spacer having the generic formula
—(CH₂)ₙ— or —[(CH₂)ₘ—O)]ₙ—, wherein m is 1 to 100 and n is 1 to 50,000, and bound thereto —OSO₃H or —OSO₃Na groups,
so that a degree of sulfation of 1 to 100% is obtained,
and
c) a molecular weight of 200 to 5,000,000 g/mol,
or
a method for the diagnosis of an anti-inflammatory disease, comprising bringing a dendritic polyglycerol sulphate loaded with signalling molecules or having signalling molecules bound thereto together with a subject having said anti-inflammatory disease, wherein the dendritic polyglycerol sulphate has the following characteristics
a) a polymeric polyglycerol core, composed of repeated units of glycerin with the formula (RO—CH₂)₂CH—OR
on a multifunctional starter molecule, which is a polyhydroxy compound having 1 to 1,000 OH groups,
wherein R═H or further glycerin units,
the core having a branching degree of 0 to 100% and
an average molecular weight of 100 to 1,000,000 g/mol,
b) the substitution of one or more OH groups of the glycerin units with —OSO₃H or —OSO₃Na groups
or the attachment of an oligomeric spacer at one or more OH groups of the glycerin units,
the oligomeric spacer having the generic formula
—(CH₂)ₙ— or —[(CH₂)ₘ—O)]ₙ—, wherein m is 1 to 100 and n is 1 to 50,000, and bound thereto —OSO₃H or —OSO₃Na groups,
so that a degree of sulfation of 1 to 100% is obtained,
and
c) a molecular weight of 200 to 5,000,000 g/mol,
or
a method for binding of proteins, wherein the proteins are selectins or chemokines, comprising bringing a dendritic polyglycerol sulfate, optionally loaded with signalling molecules or having signalling molecules bound thereto, together with said proteins, wherein the dendritic polyglycerol sulphate is characterized by:
a) a polymeric polyglycerol core, composed of repeated units of glycerin with the formula (RO—CH₂)₂CH—OR
on a multifunctional starter molecule, which is a polyhydroxy compound having 1 to 1,000 OH groups,
wherein R═H or further glycerin units,
the core having a branching degree of 0 to 100% and
an average molecular weight of 100 to 1,000,000 g/mol,
b) the substitution of one or more OH groups of the glycerin units with —OSO₃H or —OSO₃Na groups
or the attachment of an oligomeric spacer at one or more OH groups of the glycerin units,
the oligomeric spacer having the generic formula
—(CH₂)ₙ— or —[(CH₂)ₘ—O)]ₙ—, wherein m is 1 to 100 and n is 1 to 50,000, and bound thereto —OSO₃H or —OSO₃Na groups,
so that a degree of sulfation of 1 to 100% is obtained,
and
c) a molecular weight of 200 to 5,000,000 g/mol.
